(19) United States
(12) Patent Application Publication
(10) Pub. No.: US 2024/0390233 A1
(43) Pub. Date: Nov. 28, 2024

(54) Bolus Feed Delivery System

(71) Applicant: Avent, Inc., Alpharetta, GA (US)

(72) Inventors: James M. Takeuchi, Woodstock, GA (US); Sarah M. Sarkissian, Roswell, GA (US); Jeffrey J. Farmer, Roswell, GA (US)

(21) Appl. No.: 18/322,648

(22) Filed: May 24, 2023

(51) Int. Cl. A61J 15/00; A61J 1/14; A61M 39/10

(52) U.S. Cl. CPC A61J 15/0015; A61J 1/1412; A61J 1/1481; A61J 15/0053; A61M 39/10; A61M 2039/1077

(57) ABSTRACT

A bolus feed delivery system is provided and includes a coupling configured to couple to a source of enteral nutrition; and a tube having a proximal end and a distal end, and a lumen extending between the proximal end and the distal end. Specifically, the proximal end is coupled to the nutrition adapter downstream of the coupling. The bolus delivery feeding system further includes a feeding tube adapter coupled to the distal end of the tube. Specifically, the feeding tube adapter includes a coupling configured to be coupled to a feeding tube receiving port. Further, the feeding tube adapter includes a lumen fluidly coupling the lumen of the tube with a lumen of the receiving port, and the lumen extends continuously in a linear direction. Further, the nutrition adapter, the tube, and the feeding tube adapter are formed as one piece.

### FIELD OF THE INVENTION

The subject matter of the present invention relates generally to a custom extension set for administration of a bolus feed via enteral feeding.

### BACKGROUND

There are many issues encountered by users of enteral feeding devices today. For example, although enteral feeding can be provided without a pump by employing a bolus or gravity feed, the administration of a bolus or gravity feed to an enteral feeding tube is often not discrete and can be cumbersome. Specifically, enteral feeding patients who bolus or gravity feed must either hold a syringe filled with nutrition at a height disposed vertically above the feeding tube, hold a bag filled with nutrition above the feeding tube, or hang a bag on an intravenous (IV) pole above the feeding tube and then wait for the nutrition to enter the feeding tube using gravity.

These existing methods for administration of a bolus feed often require nutritional fluid to be transferred from a nutrition source (e.g., carton) to a syringe or bag that can be coupled to a tube set for delivery of the feed to the feeding tube, thereby requiring more supplies and introducing potential sources of contamination to the nutritional fluid prior to delivery of the feed. Moreover, these existing methods are not very discrete, and may still cause a user or patient to feel stigmatized or isolated. In addition, these methods for administration of a bolus feed often require the assistance of a caregiver, and can be cumbersome with the amount of supplies needed. Current options for bolus feed are not ideal for patients on-the-go, away from home or in a clinical setting.

Accordingly, improved systems for administration of an enteral feeding bolus are desired in the art. In particular, an extension set which provides direct connection from a feed source to a patient's feeding tube without the need for any additional extension set or connections would be advantageous.

### SUMMARY

Objects and advantages of the invention will be set forth in part in the following description, or may be obvious from the description, or may be learned through practice of the invention.

In accordance with one aspect, a bolus feed delivery system for enteral feeding is provided. The bolus delivery feed system includes a nutrition adapter including a coupling configured to couple to a source of enteral nutrition; and a tube having a proximal end and a distal end, and a lumen extending between the proximal end and the distal end. Specifically, the proximal end is coupled to the nutrition adapter downstream of the coupling. The bolus delivery feeding system further includes a feeding tube adapter coupled to the distal end of the tube. Specifically, the feeding tube adapter includes a coupling configured to be coupled to a feeding tube receiving port. Further, in the same aspect, the feeding tube adapter includes a lumen fluidly coupling the lumen of the tube with a lumen of the receiving port, and the lumen extends continuously in a linear direction. Further, in the same aspect, the nutrition adapter, the tube, and the feeding tube adapter are formed as one piece.

In one embodiment of the bolus feed delivery system, an angle exists between a proximal end of the lumen of the feeding tube adapter and a distal end of the lumen of the feeding tube adapter in a range from about 165 degrees to about 195 degrees.

In another embodiment of the bolus feed delivery system, the angle is about 180 degrees.

In a further embodiment of the bolus feed delivery system, the bolus feed delivery system further includes a solvent or adhesive bond between the proximal end of the first tube and the nutrition adapter.

In an additional embodiment of the bolus feed delivery system, the bolus feed delivery system further includes a solvent or adhesive bond between the distal end of the tube and the feeding tube adapter.

In yet another embodiment of the bolus feed delivery system, the feeding tube adapter coupling includes a male coupling configured to be received within a female coupling of the feeding tube receiving port. Further, the male coupling includes at least one rib protruding from an external surface of the male coupling.

In still another embodiment of the bolus feed delivery system, the bolus delivery system further includes a flow controller.

In yet a further embodiment of the bolus feed delivery system, the flow controller includes a clamp including an aperture. Further, the tube extends through the aperture, and the clamp is configured to restrict flow of fluid through the tube.

In still a further embodiment of the bolus feed delivery system, the bolus feed delivery system further includes an inline access port attached to the tube.

In another additional embodiment of the bolus feed delivery system the inline access port is formed as one piece with the nutrition adapter, the tube, and the feeding tube adapter.

In a further additional embodiment of the bolus feed delivery system, the nutrition adapter includes a first inlet port configured to couple with the source of enteral nutrition and a second inlet port configured to couple with a secondary fluid source. Furthermore, the nutrition adapter further includes a tethered cap configured to seal the second inlet port.

In yet another additional embodiment of the bolus feed delivery system, the source of enteral nutrition includes an original source of nutrition. Further, the original source of nutrition is a single-use, disposable nutrition package.

In accordance with another aspect, a kit for delivery of a bolus feed via enteral feeding is provided. The kit includes a bolus feed extension set including a nutrition adapter comprising a coupling configured to couple to a source of enteral nutrition; a tube having a proximal end and a distal end, and a lumen extending between the proximal end and the distal end. Specifically, the proximal end is coupled to the nutrition adapter downstream of the coupling. The bolus feed extension set of the kid further includes a feeding tube adapter coupled to the distal end of the tube, wherein the feeding tube adapter includes a coupling configured to be coupled to a feeding tube receiving port. Further, in the same aspect, the feeding tube adapter includes a lumen fluidly coupling the lumen of the tube with a lumen of the receiving port, and the lumen extends continuously in a linear direction. Further, in the same aspect, the nutrition adapter, the tube, and the feeding tube adapter are formed as one piece. Further, in the same aspect, the kit includes a nutrition source; and a support for the nutrition source. Specifically, the support is configured to suspend the nutrition source vertically above the feeding tube receiving port to enable gravity feeding from the nutrition source to the feeding tube receiving port.

In one embodiment of the kit, the support includes a garment to be worn by a patient.

In accordance with another aspect, a method for delivering a bolus feed to a patient is provided. The method includes providing a bolus feed delivery system for enteral feeding including a nutrition adapter comprising a coupling configured to couple to a source of enteral nutrition; a tube having a proximal end and a distal end, and a lumen extending between the proximal end and the distal end where the proximal end is coupled to the nutrition adapter downstream of the coupling; a feeding tube adapter coupled to the distal end of the tube where the feeding tube adapter includes a coupling configured to be coupled to a feeding tube receiving port where the feeding tube adapter includes a lumen fluidly coupling the lumen of the tube with a lumen of the receiving port where the lumen extends continuously in a linear direction where the nutrition adapter, the tube, and the feeding tube adapter are formed as one piece. In the same aspect, the method further includes joining the coupling of the nutrition adapter to the source of enteral nutrition. In the same aspect, the method further includes joining the coupling of the feeding tube adapter to the feeding tube receiving port. In the same aspect, the method further includes delivering the bolus feed to the patient from the source of enteral nutrition through the bolus feed delivery system.

These and other features, aspects, and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a perspective view of a bolus feed delivery system according to one particular embodiment of the present invention;

FIG. 2 illustrates a perspective view of a bolus feed delivery system according another particular embodiment of the present invention;

FIG. 3 illustrates a perspective view of a bolus feed delivery system being positioned for coupling to a source of enteral nutrition according to a particular embodiment of the present invention;

FIG. 4 illustrates a perspective view of a bolus feed delivery system being positioned for coupling to a feeding tube according to a particular embodiment of the present invention;

FIG. 5 illustrates a perspective view of a bolus feed delivery system, particularly illustrating an inline access port provided with the bolus feed delivery system;

FIG. 6 illustrates a perspective view of a kit for delivery of a bolus feed via enteral feeding according to a particular embodiment of the present invention; and

FIG. 7 illustrates a flow chart of a method for delivering a bolus feed to a patient in accordance with a particular embodiment of the present disclosure.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents. The detailed description uses numerical and letter designations to refer to features in the drawings. Like or similar designations in the drawings and description have been used to refer to like or similar parts of the invention.

As used herein, the terms “first”, “second”, and “third” may be used interchangeably to distinguish one component from another and are not intended to signify location or importance of the individual components. The singular forms “a,” “an,” and “the” include plural references unless the context clearly dictates otherwise. The terms “coupled,” “fixed,” “attached to,” and the like refer to both direct coupling, fixing, or attaching, as well as indirect coupling, fixing, or attaching through one or more intermediate components or features, unless otherwise specified herein. As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Terms of approximation, such as “about,” “generally,” “approximately,” or “substantially,” include values within ten percent greater or less than the stated value. When used in the context of an angle or direction, such terms include within ten degrees greater or less than the stated angle or direction. For example, “generally vertical” includes directions within ten degrees of vertical in any direction, e.g., clockwise or counter-clockwise. Further, when a plurality of ranges are provided, any combination of a minimum value and a maximum value described in the plurality of ranges are contemplated by the present invention. For example, if ranges of “from about 20% to about 80%” and “from about 30% to about 70%” are described, a range of “from about 20% to about 70%” or a range of “from about 30% to about 80%” are also contemplated by the present invention.

As will be used herein, the terms proximal and distal each refer to a point of reference in relation to a particular object. Specifically, the term proximal is defined as being situated nearer the object of reference, while the term distal is defined as being situated further from the object of reference. As described herein, the object of reference is the source of nutrition. Thus, an object that is proximal is closer to the source of nutrition, while an object that is distal is further from the source of nutrition.

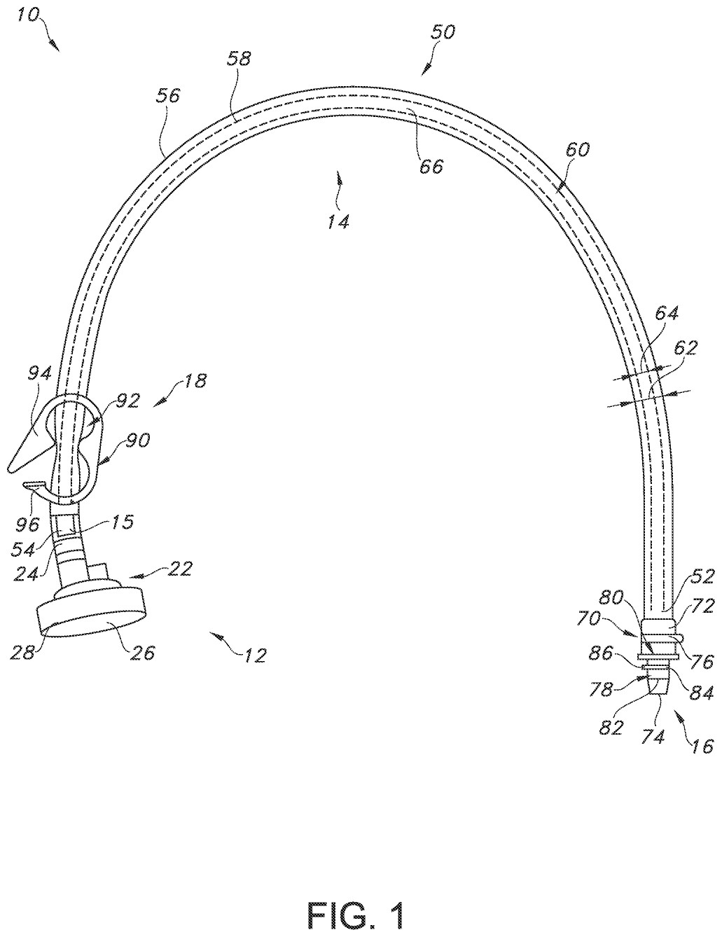

Generally speaking, the present invention is directed to a bolus feed delivery system having a nutrition source adapter, a tube, and a feeding tube adapter formed as a single integrated extension set. The bolus feed delivery system extension set includes a built-in adapter that connects directly to the container where the nutrition is stored and connects directly to the feeding tube port, thereby forming an all-in-one giving set and extension set that removes the need to transfer nutrition from the nutrition source container to a syringe or a giving set bag for feeding. Additionally, the present invention is directed to a kit for delivery of a bolus feed that includes a bolus feed delivery system and a support, e.g., a garment, to hold a source of nutrition above the patient's feeding tube to enable administration of a bolus feed on-the-go. The specific features of the bolus feed delivery system and kit of the present invention may be better understood with reference to FIGS. 1-5.

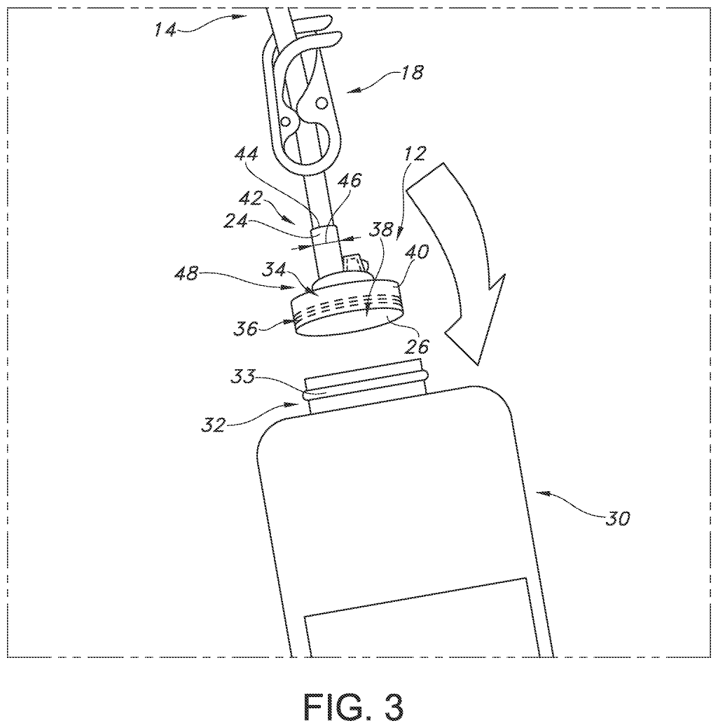

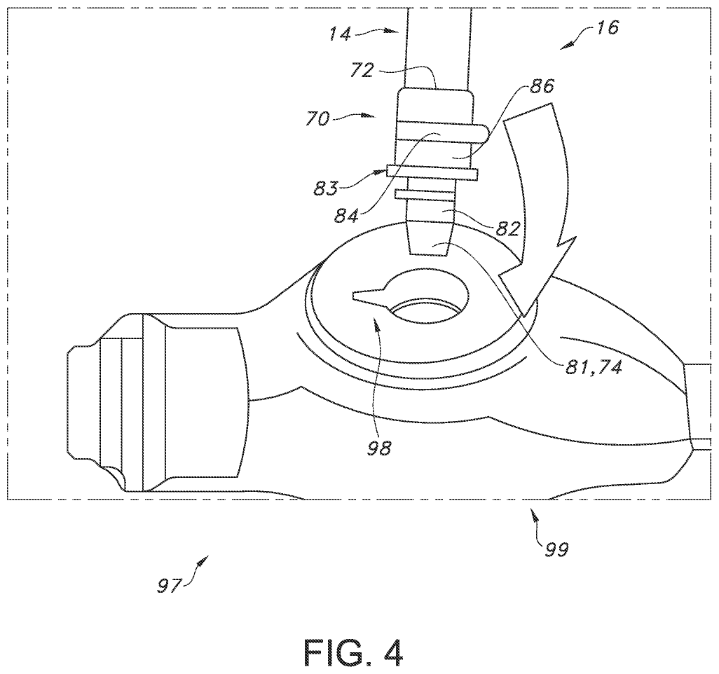

Referring now to FIG. 1, one embodiment of a bolus feed delivery system 10 is shown. The bolus feed delivery system 10 includes a nutrition adapter 12 configured to be coupled to a nutrition source (see FIG. 3), a tube 14, and a feeding tube adapter 16 configured to be coupled directly with an indwelling feeding tube 97 (see FIG. 4). The delivery system 10 may additionally include a flow restriction mechanism 18 configured to enable or occlude flow of nutrition through the tube 14. For instance, the flow restriction mechanism 18 may be formed as a clamp.

The nutrition adapter 12 has a body 22 extending from a distal end 24 to a proximal end 26. The proximal end 26 includes a nutrition connector 28 configured and designed to complementarily attach directly to a connector 32 of source of nutrition 30 (see FIG. 3). The source of nutrition 30 may be a bag, a carton formed from plastic, cardboard or other suitable material, a reusable nutrition container (e.g., formed from plastic, thermoplastic or glass), or any other container for nutrition having a cooperating connector. In particular, the source of nutrition 30 to which the nutrition adapter 12 is designed to be coupled is intended to be an original source of nutrition, e.g., nutritional formula in a single-use package for bolus feeding as packaged by a manufacturer or source; however, reusable containers may also be coupled to the nutrition adapter 12. For instance, the proximal end 26 may include a collar 34 having threads 36 formed on an inner surface 38 or outer surface 40 of the collar 34. In other aspects of the present invention, the nutrition connector 28 may include a spike adapter (not shown) having a conical or cylindrical shape configured to be inserted into a source of nutrition 30. In still other aspects of the present invention, the nutrition connector 28 may include a push-on plug body configured for coupling to a plug receiver of a source of nutrition 30. The connector 32 of the source of nutrition 30 may include matching coupling features. For example, if the collar 34 has threads 36, the connector 32 may also include matching threads 33 that correspond with the threads 36.

At the distal end 24 of the nutrition adapter 12, the body 22 includes a tube coupling 42. The tube coupling 42 includes a cylindrical outer surface 44 having a diameter 46 configured to correspond to an inner diameter or outer diameter of the tube 14, as described in further detail below. Thus, the tube coupling 42 may be inserted into the tube 14 or the tube coupling 42 may surround an outer diameter of the tube 14. In either instance, a proximal end 54 of the tube 14 is coupled to the nutritional adapter 12 downstream of the tube coupling 42. Further, the tube coupling 42 and the tube 14 may be permanently, i.e., irremovably, coupled together. For instance, the tube coupling 42 and the tube 14 may be solvent bonded, adhesively bonded, or coupled by any other suitable means such that the tube coupling 42 and the tube 14 may not be separated without destroying the integrity of the tube 14. As a result of this bonding, the tube coupling 42 and the tube 14 may be formed as one piece when used in the bolus feed delivery system 10.

In any instance, the body 22 of the nutrition adapter 12 includes a nutrition lumen 48 extending therethrough from the nutrition connector 28 to the tube coupling 42. In this arrangement, when the nutrition adapter 12 is connected to a source of nutrition 30, the nutrition fluid can flow from the source of nutrition 30 through the length of the body 22 from the nutrition connector 28 through the tube coupling 42 and into the tube 14.

The body 22 of the nutrition adapter 12 may be formed from any suitable thermoplastic material, including but not limited to acrylonitrile butadiene styrene (ABS), polycarbonate, polypropylene, and other materials. A suitable thermoplastic material for the body 22 is rigid and can be formed, e.g., by molding, into the shape of the body 22.

The tube 14 has a body 50 that extends from a distal end 52 to a proximal end 54. The body 50 of the tube 14 includes an outer wall 56 and an inner wall 58. The inner wall 58 may be concentrically disposed within the outer wall 56. A lumen 60 is defined by the inner wall 58 and extends along the length of the body 50 from the distal end 52 to the proximal end 54. The body 50 has an outer diameter 62 defined by the outer wall 56 and an inner diameter 64, also defined as a lumen diameter, defined by the inner wall 58. The body 50 of the tube 14 may be formed from any suitable flexible material used for delivery of nutritional fluid, including but not limited to flexible polyvinyl chloride (PVC), silicone, polyurethane, and other thermoplastic elastomers.

A flow rate of nutrition through the tube 14 can be determined at least in part by the inner diameter 64 of the tube 14 and a length 66 extending from the distal end 52 to the proximal end 54 of the tube. In general, the larger the inner diameter 64, the higher the flow rate of nutrition through the tube 14. However, the flow rate of nutrition through the tube 14 may be influenced by additional factors, including but not limited to the length and diameter of the nutrition lumen 48 through the body 22 of the nutrition adapter 12, the presence of a flow restriction mechanism 18, the viscosity and/or composition of the nutrition delivered through the tube 14, and other factors.

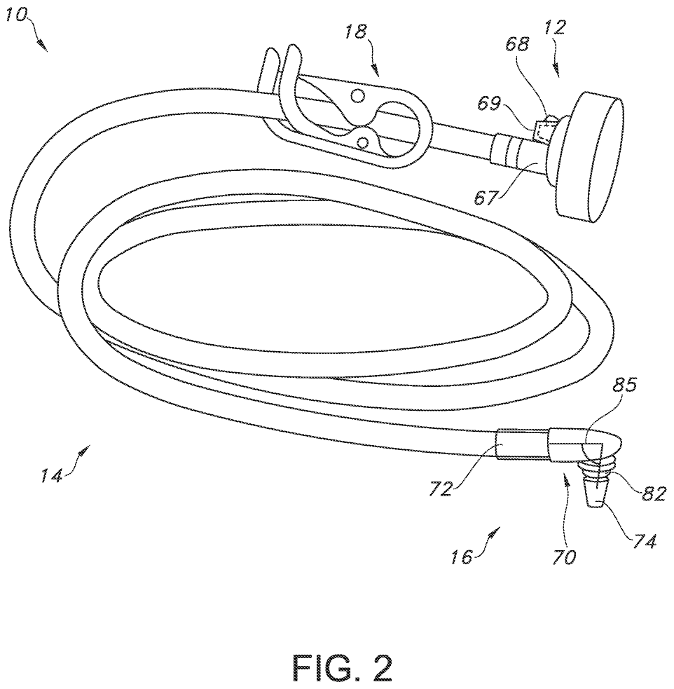

The nutrition adapter 12 may also include multiple ports such that multiple sources of nutrition or other fluids may be connected to the tube 14. For example, referring to FIG. 2, the nutrition adapter 12 may include a first inlet port 67 configured to couple with the source of enteral nutrition 30 and a second inlet port 68 configured to couple with a secondary fluid source (not shown). The secondary fluid source may be another source of enteral nutrition 30, or alternatively, the secondary fluid source may be a medicine such that feeding and medicating are accomplished with the same bolus feed delivery system 10. In addition, the nutrition adapter 12 may further include a tethered cap 69 configured to seal the second inlet port 68. By providing the tethered cap 69, second inlet port 68 may be kept sterile until it is connected to the secondary fluid source.

Referring back to FIG. 1, a feeding tube adapter 16 is coupled to the distal end 52 of the tube 14. The feeding tube adapter 16 has a body 70 that extends from a proximal end 72 to a distal end 74. The body 70 is coupled to the distal end 52 of the tube 14 at the proximal end 72 of the feeding tube adapter 16. For instance, the proximal end 72 may include a collar 76 configured to be inserted within the lumen 60 of the tube 14 and/or configured to receive the outer wall 56 of the tube 14 therein within the collar 76. Similarly to the connection between the tube 14 and the nutrition adapter 12 at the proximal end 54 of the tube 14, the body 70 of the feeding tube adapter 16 and the tube 14 may be permanently, i.e., irremovably, coupled together. For instance, the collar 76 and the tube 14 may be solvent bonded, adhesively bonded, or coupled by any other suitable means such that the body 70 and the tube 14 may not be separated without destroying the integrity of the tube 14. Consequently, like the tube coupling 42 and the tube 14, the tube 14 and body 70 may be formed as one piece when used in the bolus feed delivery system 10.

Further, the bolus feed delivery system 10 can be formed as a single unit. Stated differently, the nutrition source adapter 12, the tube 14, and the feeding tube adapter 16 can be irremovably coupled together such that none of the components are separated from each other without destroying the system 10 and rendering the system 10 inoperable and unsuited for its intended purpose of the delivery of a bolus feed to a feeding tube. For example, the bolus feed delivery system 10 can be welded together using a technique such as ultrasonic welding or thermal welding.

At the distal end 74 of the body 70, the feeding tube adapter 16 includes a feeding tube coupling 78. The feeding tube coupling 78 is configured to be inserted directly into a receiving port 99 (see FIG. 4) of an indwelling feeding tube 97 having a lumen. A feeding tube adapter lumen 80 extends through the body 70 from the proximal end 72 to the distal end 74 to fluidly couple the feeding tube adapter 16 with the tube 14 such that nutrition may be delivered from the tube 14 to the feeding tube 97 when the coupling 78 is inserted within the receiving port of the feeding tube 97 (see FIG. 4).

The feeding tube coupling 78 may comprise a stem 82 configured to be inserted into the receiving port 99 of the indwelling feeding tube 97. The stem 82 may form a male connector or coupling configured to be inserted into a female receiving coupling or port 99 of the indwelling feeding tube 97. The stem 82 may have a cylindrical cross-sectional shape or any other rounded cross-sectional shape. The stem 82 may have a tapered proximal end 84 having a diameter that tapers toward the terminal distal end 81 of the stem 82.

The body 70 can extend in a continuous longitudinal or linear direction such that there is a generally straight line extending between the proximal end 72 and the distal end 74 of the body 70. Stated differently, a generally straight line may extend from the proximal end 72 through the stem 82. For instance, the stem 82 can extend along an axis parallel with the body 70 at an angle of from about 165 degrees to about 195 degrees, such as from about 170 degrees to about 190 degrees, for instance from about 175 degrees to about 185 degrees, for instance at 180 degrees, with respect to the proximal end 72 of the body 70. In one aspect of the present invention, the distal end 74 of the lumen 80 may extend at an angle of about 180 degrees from the proximal end 72 of the lumen 80, i.e., the lumen 80 extends in a generally straight line. The present inventors have found that providing a feeding tube adapter 16 having a straight or substantially straight configuration provides a temporary connection intended to quickly deliver a bolus of nutritional fluid to the patient and then be disconnected from the patient. Such a configuration may be suitable for bolus delivery feeding utilizing the bolus delivery feed system 10.

In another aspect of the present invention, as illustrated in FIG. 2, the stem 82 and distal end 74 of the body 70 may extend at an angle with respect to the proximal end 72. For instance, the stem 82 may extend from an axis parallel to the body 70 at an angle in a range from about 70 degrees to about 110 degrees with respect to the proximal end 72. More particularly, the proximal end 72 and distal end 74 may be generally perpendicular to each other, in other words, at about a 90 degree angle relative to each other, as shown in FIG. 2.

As shown in FIG. 4, the body 70 may include one or more mating features 86 configured to removably couple with complementary mating or locking features of the receiving port 99 of the feeding tube 97. For example, the body 70 may include one or more ribs or protrusions configured to be received within a complementary receiving port 99. For example, the ribs may protrude from an external surface 83 of the stem 82. As stated hereinabove, the receiving port 99 may also include mating features 98. For example, the mating features may include a groove that is sized for the insertion of either the stem 82 or the mating features 86 of the body 70.

As shown in FIG. 1, a flow restriction mechanism 18 may be coupled to the tube 14 to restrict or control the flow of fluid from the nutrition source through the tube 14. For instance, the flow restriction mechanism 18 may be formed as a clamp 90 as shown in FIG. 1. The clamp 90 may include at least one aperture 92 through which the tube body 50 extends. Further, the clamp 90 may include at least one restriction member 94 configured to be positioned in an open or closed configuration. In the open configuration, fluid may flow through the tube 14 without restriction from the flow restriction mechanism 18. In the closed configuration, the restriction member 94 may be compressed against the tube body 50 and held in place by a locking member 96. The restriction member 94 may thereby compress the tube body 50 to prevent and/or restrict flow of fluid through the lumen 60 at the point at which the tube body 50 is compressed. In some aspects, the restriction member 94 may full prevent flow of fluid through the lumen 60. In other aspects of the present invention, the restriction member 94 may restrict or limit, i.e., slow, the flow of fluid through the lumen 60 to decrease the flow rate of fluid through the tube 14.

In addition to or as an alternative to the push-style clamp 90, the flow restriction mechanism 18 may incorporate a roller-style clamp. A roller-style clamp may enable varying degrees of adjustment in the amount the tubing is compressed, and thus, modulate fluid flow through the tube. Still further, a rotary style flow restriction mechanism that inserts connectors of varying sizes inline to the tubing may be provided to modulate fluid flow through the tube 14. These connectors could be formed from a variety of designs including, but not limited to, tubing segments and pieces of rigid plastic. A variety of lengths and sizes, e.g. diameter, of connectors for a rotary style flow restrictor could be offered to achieve different amounts of flow. In such an arrangement, a user could rotate a dial (or other mechanism) that adjusts the flow path of the fluid through one of the connectors, and thus, change the flow rate of fluid through the device.

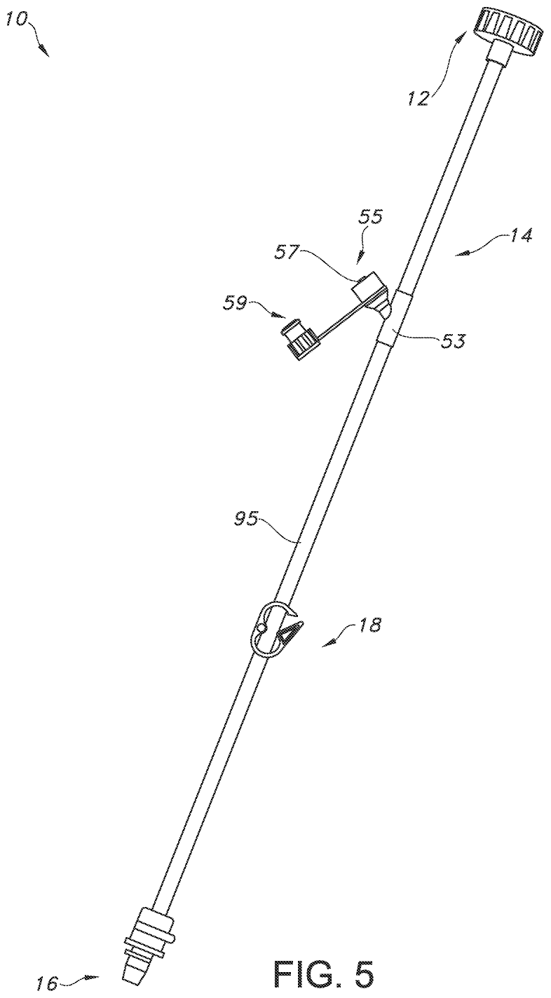

Referring now to FIG. 5, one embodiment of a bolus feed delivery system 10 is shown, particularly illustrating an inline access port 51 provided with the bolus feed delivery system 10. As shown, the inline access port 51 may be attached to the tube 14 downstream of the nutrition source adapter 12. Specifically, the inline access port 51 may include a tubular portion 53 that the tube 14 is inserted into or attached with. The inline access port 51 may also include a branched portion 55, such as a Y-shaped portion, that allows for the provision of a second source of fluid such as nutrition or medicine. The inline access port 51 may also allow for the flushing of the tube 14 if any nutrition or other matter remains in the tube 14. The inline access port 51 may also allow for the decompressing or venting of the patient's stomach that is using the bolus feed delivery system 10. To achieve these features, the branched portion 55 may include a secondary connector 57. The secondary connector 57 may have a variety of shapes and configurations to attach to whatever the second source of fluid is provided. For example, the secondary connector 57 may be a threaded or screw-on connection or a press-fit connection.

In addition, the inline access port 51 may be solvent bonded, adhesively bonded, or coupled by any other suitable means to the tube 14 such that the inline access port 51 is inseparable from the tube 14. Further, like the nutrition source adapter 12, the tube 14 and the feeding tube adapter 16, the inline access port 51 may be irremovably coupled from the nutrition source adapter 12, the tube 14, and the feeding tube adapter 16 such that none of the components are separated from each other without destroying the system 10 and rendering the system 10 inoperable and unsuited for its intended purpose of the delivery of a bolus feed to a feeding tube. Consequently, the nutrition source adapter 12, the tube 14, the feeding tube adapter 16, and the inline access port 51 may all be formed as one piece when used in the bolus feed delivery system 10.

In another embodiment, a second tube 95 may be provided that connects between the inline access port 51 and the feeding tube adapter. If a second tube 95 is provided, the nutrition source adapter 12, the tube 14, the feeding tube adapter 16, the inline access port 51, and the second tube 95 may all be formed as one piece when used in the bolus feed delivery system 10 as described above.

In addition, as shown, the inline access port 51 may include a tethered cap 59. The tethered cap 59 may be attached to the branched portion 55 of the inline access port 51. Further, the tethered cap 59 may allow for the sealing of the inline access port 51, if it is not currently being used.

In addition, as shown, if the inline access port 51 is provided, the flow restriction mechanism 18 may be provided downstream of both the nutrition adapter 12 and the inline access port 51. By placing the flow restriction mechanism 18 downstream, the flow restriction mechanism 18 may restrict or prevent the flow of nutrition from the nutrition adapter 12 or the inline access port 51.

A kit 100 for delivery of a bolus feed via enteral feeding may further be provided. The kit 100 can include the bolus feed delivery system 10 described above and a support 110 configured to support, e.g., hold in place, a nutrition source 120. The nutrition source 120 may further be included in the kit 100. The support 110 can be in the form of a garment to be worn by a patient or any other suitable support configured to engage with the nutrition source 120. For instance, an IV pole or other mount for supporting the nutrition source 120 at a height above the patient's feeding tube may be provided as a support 110.

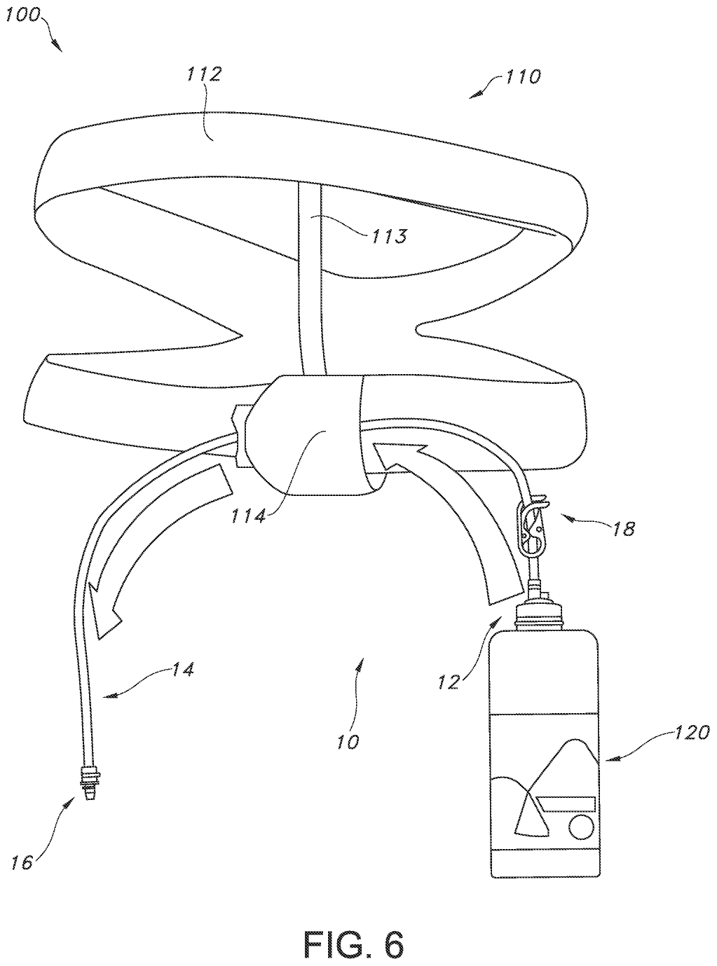

As shown in FIG. 6, the support 110 may include a garment 112 to be worn by a patient. The garment 112 can be, e.g., a vest, a belt, or a harness having one or more straps to secure the garment 112 to the patient. The garment 112 may include a crisscrossed shape to be better secured to the patient. The garment 112 may also include a supporting portion 113 that spans across the crisscrossed shape. The supporting portion 113 may better secure the crisscrossed shape of the garment 112 to the patient while preventing a nutrition source 120 held within the garment 112 from sagging downward due to gravity.

The garment 112 may include one or more pockets 114 configured to hold or contain the nutrition source 120. The pocket(s) 114 can be disposed at any suitable portion of the garment that is configured to be suspended or positioned vertically above the patient's feeding tube in order to enable the nutritional fluid to flow through the tube 14 to the feeding tube by gravity. For example, the pocket(s) 114 may be placed at the bottom of the garment as shown. The garment 112 may be worn either over or concealed beneath a patient's clothing. By including a support 110 such as a wearable garment 112 to hold the nutrition source 120 above the feeding tube to enable a gravity-assisted bolus feed, the kit 100 may advantageously enable a patient to conduct a bolus feed on-the-go, i.e., away from home or a clinical setting.

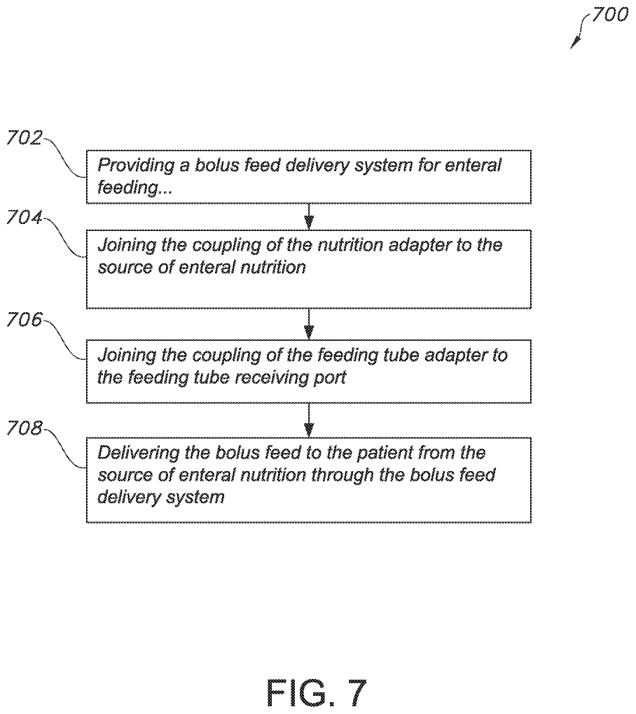

As shown in FIG. 7, the present invention may be further directed to a method for delivering a bolus feed to a patient. The method 700 may be executed using the bolus feed delivery system 10 as detailed above. At step 702, the method includes providing a bolus feed delivery system, such as bolus feed delivery system 10, for enteral feeding. Next, at step 704, the method includes joining the coupling of the nutrition adapter to the source of enteral nutrition. Next, at step 706, the method includes joining the coupling of the feeding tube adapter to the feeding tube receiving port. Next, at step 708, the method includes delivering the bolus feed to the patient from the source of enteral nutrition through the bolus feed delivery system.

An exemplary technical effect of the methods, systems, and apparatus described herein includes at least one of, but are not limited to: a) removing the need to transfer enteral nutrition from its original source to a separate container for enteral feeding, b) improving efficiency of delivery of a bolus feed, especially in an outpatient setting, and c) improving ease of preparation of an enteral feed for enteral feeding.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

What is claimed is:

1. A bolus feed delivery system for enteral feeding comprising:
a nutrition adapter comprising a coupling configured to couple to a source of enteral nutrition;
a tube having a proximal end and a distal end, and a lumen extending between the proximal end and the distal end, wherein the proximal end is coupled to the nutrition adapter downstream of the coupling;
a feeding tube adapter coupled to the distal end of the tube, wherein the feeding tube adapter comprises a coupling configured to be coupled to a feeding tube receiving port,
wherein the feeding tube adapter comprises a lumen fluidly coupling the lumen of the tube with a lumen of the receiving port, wherein the lumen extends continuously in a linear direction, and
wherein the nutrition adapter, the tube, and the feeding tube adapter are formed as one piece.

2. The bolus feed delivery system of claim 1, wherein an angle between a proximal end of the lumen of the feeding tube adapter and a distal end of the lumen of the feeding tube adapter is in a range from about 165 degrees to about 195 degrees.

3. The bolus feed delivery system of claim 2, wherein the angle is about 180 degrees.

4. The bolus feed delivery system of claim 1, further comprising a solvent or adhesive bond between the proximal end of the first tube and the nutrition adapter.

5. The bolus feed delivery system of claim 1, further comprising a solvent or adhesive bond between the distal end of the tube and the feeding tube adapter.

6. The bolus feed delivery system of claim 1, wherein the feeding tube adapter coupling comprises a male coupling configured to be received within a female coupling of the feeding tube receiving port, wherein the male coupling comprises at least one rib protruding from an external surface of the male coupling.

7. The bolus feed delivery system of claim 1, further comprising a flow controller.

8. The bolus feed delivery system of claim 7, wherein the flow controller comprises a clamp comprising an aperture, wherein the tube extends through the aperture, further wherein the clamp is configured to restrict flow of fluid through the tube.

9. The bolus feed delivery system of claim 1 further comprising an inline access port attached to the tube.

10. The bolus feed delivery system of claim 9, wherein the inline access port is formed as one piece with the nutrition adapter, the tube, and the feeding tube adapter.

11. The bolus feed delivery system of claim 1, wherein the nutrition adapter comprises a first inlet port configured to couple with the source of enteral nutrition and a second inlet port configured to couple with a secondary fluid source, further comprising a tethered cap configured to seal the second inlet port.

12. The bolus feed delivery system of claim 1, wherein the source of enteral nutrition comprises an original source of nutrition, wherein the original source of nutrition is a single-use, disposable nutrition package.

13. A kit for delivery of a bolus feed via enteral feeding, the kit comprising:
a bolus feed extension set comprising:
a nutrition adapter comprising a coupling configured to couple to a source of enteral nutrition;
a tube having a proximal end and a distal end, and a lumen extending between the proximal end and the distal end, wherein the proximal end is coupled to the nutrition adapter downstream of the coupling;
a feeding tube adapter coupled to the distal end of the tube, wherein the feeding tube adapter comprises a coupling configured to be coupled to a feeding tube receiving port,
wherein the feeding tube adapter comprises a lumen fluidly coupling the lumen of the tube with a lumen of the receiving port, wherein the lumen extends continuously in a linear direction, and
wherein the nutrition adapter, the tube, and the feeding tube adapter are formed as one piece;
a nutrition source; and
a support for the nutrition source, wherein the support is configured to suspend the nutrition source vertically above the feeding tube receiving port to enable gravity feeding from the nutrition source to the feeding tube receiving port.

14. The kit of claim 13, wherein an angle between a proximal end of the lumen of the feeding tube adapter and a distal end of the lumen of the feeding tube adapter is in a range from about 165 degrees to about 195 degrees.

15. The kit of claim 13, further comprising a solvent or adhesive bond between the proximal end of the first tube and the nutrition adapter and a solvent or adhesive bond between the distal end of the tube and the feeding tube adapter.

16. The kit of claim 13, wherein the feeding tube adapter coupling comprises a male coupling configured to be received within a female coupling of the feeding tube receiving port, wherein the male coupling comprises at least one rib protruding from an external surface of the male coupling.

17. The kit of claim 13, further comprising an inline access port attached to the tube, wherein the inline access port is formed as one piece with the nutrition adapter, the tube, and the feeding tube adapter.

18. The kit of claim 13, wherein the nutrition adapter comprises a first inlet port configured to couple with the source of enteral nutrition and a second inlet port configured to couple with a secondary fluid source, further comprising a tethered cap configured to seal the second inlet port.

19. The kit of claim 13, wherein the support comprises a garment to be worn by a patient.

20. A method for delivering a bolus feed to a patient, the method comprising:
providing a bolus feed delivery system for enteral feeding comprising:
a nutrition adapter comprising a coupling configured to couple to a source of enteral nutrition,
a tube having a proximal end and a distal end, and a lumen extending between the proximal end and the distal end, wherein the proximal end is coupled to the nutrition adapter downstream of the coupling,
a feeding tube adapter coupled to the distal end of the tube, wherein the feeding tube adapter comprises a coupling configured to be coupled to a feeding tube receiving port, wherein the feeding tube adapter comprises a lumen fluidly coupling the lumen of the tube with a lumen of the receiving port, wherein the lumen extends continuously in a linear direction, and
wherein the nutrition adapter, the tube, and the feeding tube adapter are formed as one piece;
joining the coupling of the nutrition adapter to the source of enteral nutrition;
joining the coupling of the feeding tube adapter to the feeding tube receiving port; and
delivering the bolus feed to the patient from the source of enteral nutrition through the bolus feed delivery system.
